(19) United States
(12) Patent Application Publication
(10) Pub. No.: US 2015/0253860 A1
(43) Pub. Date: Sep. 10, 2015

(54) E-FIELD SENSING OF NON-CONTACT GESTURE INPUT FOR CONTROLLING A MEDICAL DEVICE

(71) Applicant: Fresenius Medical Care Holdings, Inc., Waltham, MA (US)

(72) Inventors: Thomas Merics, Antioch, CA (US); Alexander Brown, Danville, CA (US); Lee Tanenbaum, Walnut Creek, CA (US); Colin Weaver, Pleasanton, CA (US)

(73) Assignee: Fresenius Medical Care Holdings, Inc., Waltham, MA (US)

(21) Appl. No.: 14/200,156

(22) Filed: Mar. 7, 2014

(51) Int. Cl. G06F 3/01; G06F 3/0481; G06F 3/0484; A61M 1/36

(52) U.S. Cl. CPC G06F 3/017; A61M 1/367; G06F 3/04812; G06F 3/04847; A61M 2205/505; A61M 1/14

(57) ABSTRACT

A system provides non-contact electric field (E-field) gesture interfacing with a medical device, such as a dialysis machine. A E-field device provides for gesture detection and command recognition of the gesture by a user, such as a health care practitioner (HCP), in connection with navigating and activating screens of a dialysis machine during a dialysis treatment without requiring the HCP to physically contact the dialysis machine. With the described system, the HCP does not need to re-glove each time a change is made to an on-going dialysis treatment when interfacing with a graphical display of the dialysis machine.

### TECHNICAL FIELD

This patent application is related to processing devices and interfaces in the medical device area.

### BACKGROUND OF THE INVENTION

Hemodialysis is a process which employs a machine that includes a dialyzer to aid patients whose renal function has deteriorated to the point where their body cannot adequately rid itself of toxins. The dialyzer may include a semi-permeable membrane, the membrane serving to divide the dialyzer into two chambers. Blood is pumped through one chamber and a dialysis solution through the second. As the blood flows by the dialysis fluid, impurities, such as urea and creatinine, diffuse through the semi-permeable membrane into the dialysis solution. The electrolyte concentration of the dialysis fluid may be set so as to maintain electrolytic balance within the patient. Other purification techniques and processes may additionally be used. Hemodialysis may be generally referred to herein as “dialysis,” although it is noted that other types of dialysis exist, such a peritoneal dialysis, and it is noted that the system described herein may be used in connection with any appropriate dialysis system or similar treatment system.

Since dialysis involves removing blood from and returning blood to a patient, performing a dialysis procedure carries a degree of risk. Dialysis treatment requires monitoring of several patient vital signs and dialysis parameters during the dialysis process in order to optimize the overall efficacy of the dialysis procedure, to assess the condition of a fistula (the access to the patient's blood) and to determine the actual purification achieved. Some examples of parameters monitored and analyzed by a dialysis machine or equipment include the blood access flow rate or the rate at which blood flows out of the patient to the dialyzer, a critical parameter; and the ratio Kt/V to measure dialysis efficiency, where K is the clearance or dialysance (both terms representing the purification efficiency of the dialyzer), t is treatment time and V is the patient's total water value.

A processing device coupled to the dialysis machine may be used to manage and oversee the functions of the dialysis process and to, for example, monitor, analyze and interpret patient vital signs and dialysis parameters during a dialysis procedure. The processing device may include a display that displays information concerning the dialysis procedure and include an interface that enables configuration and control of the dialysis machine. A health care practitioner such as a nurse or a patient care technician may oversee the dialysis treatment sessions. Data provided by the dialysis machine and the processing device may aid the health care practitioner in performing his or her duties.

For various descriptions of dialysis systems and components, reference is made, for example, to U.S. Pat. No. 8,110,104 B2 to Crnkovich et al., entitled “Dialysis Systems and Related Components,” and U.S. Pat. No. 6,775,577 B2 to Crnkovich et al., entitled “Method and System for Controlling a Medical Device,” which are incorporated herein by reference. For a description of a sensor system that may be used in connection with monitoring and issuing alerts during a dialysis procedure, reference is made, for example, to U.S. Pat. No. 7,973,667 B2 to Crnkovich et al., entitled “Wetness Sensor,” which is incorporated herein by reference. For various descriptions of interfaces for dialysis systems, reference is made, for example, to U.S. Pat. No. 8,323,503 B2 to Levin et al., entitled “User Interface Processing Device” and U.S. Patent App. Pub. No. 2007/0112603 A1 to Kauthen et al., entitled “Digital Data Entry Methods and Devices,” which are incorporated herein by reference.

An operator needs to re-glove after every patient interaction during a dialysis treatment based on contact with any element of the patient care environment and such contact often involves changing a graphical screen on the dialysis machine. The user may change screens as often as once every half hour during a typical dialysis treatment. Accordingly, it would be desirable to provide a system that efficiently and effectively enables a user, such as health care practitioner overseeing the dialysis treatment, to change the screens of the dialysis machine without having to touch or otherwise physically contact the dialysis machine.

### SUMMARY OF THE INVENTION

According to the system described herein, a method for providing non-contact electric field (E-field) gesture interfacing with a medical device including operating an E-field device to enable non-contact gesture interfacing with the medical device by a user without physical contact of the user with the medical device by detecting and classifying a non-contact gesture command of the user. A command signal is received at the medical device, in which the command signal applies to a treatment being performed using the medical device, and in which the command signal corresponds to the non-contact gesture command of the user. The command signal is processed at the medical device to generate information corresponding to the treatment performed using the medical device, and the information is applied to adjust the medical device. The medical device may include a dialysis machine, and the information applied to adjust the medical device may include information that modifies dialysis treatment information displayed during a dialysis treatment. The E-field device may be integrated with the medical device, and the command signal may be integrally communicated by the E-field device to the medical device in response to the non-contact gesture command of the user. The E-field device may be implemented on a remote interface device that transmits a wireless signal to the medical device in response to the non-contact gesture command of the user. The non-contact gesture command may cause a change in the information applied to adjust the medical device, and the change in the information may include a different screen being displayed on the medical device, a section of the information being activated by the non-contact gesture command and causing a change in operation of the dialysis machine and/or a displayed cursor being illuminated by proximity detection of a hand and a location of the cursor manipulated by a position of the hand. The E-field device may include an E-field sensor that detects changes in electric field by a hand or finger of a user intruding a sensing area of the E-field sensor that detects the non-contact gesture command of the user and a gesture command recognition device that classifies the non-contact gesture command and identifies an action to be performed at the medical device.

According further to the system described herein, a non-transitory computer-readable medium stores software for providing non-contact electrical field (E-field) gesture interfacing with a medical device. The software includes executable code that operates an E-field device to enable non-contact gesture interfacing with the medical device by a user without physical contact of the user with the medical device by detecting and classifying a non-contact gesture command of the user. Executable code is provided that receives a command signal at the medical device, in which the command signal applies to a treatment being performed using the medical device, and in which the command signal corresponds to the non-contact gesture command of the user. Executable code is provided that processes the command signal at the medical device to generate information corresponding to the treatment performed using the medical device. Executable code is provided that applies the information to adjust the medical device. The medical device may include a dialysis machine, and the information applied to adjust the medical device may include information that modifies dialysis treatment information displayed during a dialysis treatment. The E-field device may be integrated with the medical device, and the command signal may be integrally communicated by the E-field device to the medical device in response to the non-contact gesture command of the user. The E-field device may be implemented on a remote interface device that transmits a wireless signal to the medical device in response to the non-contact gesture command of the user. The non-contact gesture command may cause a change in the information applied to adjust the medical device, and the change in the information may include a different screen being displayed on the medical device, a section of the information being activated by the non-contact gesture command and causing a change in operation of the dialysis machine and/or a displayed cursor being illuminated by proximity detection of a hand and a location of the cursor manipulated by a position of the hand. The E-field device may include an E-field sensor that detects changes in electric field by a hand or finger of a user intruding a sensing area of the E-field sensor that detects the non-contact gesture command of the user and a gesture command recognition device that classifies the non-contact gesture command and identifies an action to be performed at the medical device.

According further to the system described herein, a system is provided for enabling non-contact electrical field (E-field) gesture interfacing with a medical device. An electrical field (E-field) device is provided that enables non-contact gesture interfacing with the medical device by a user without physical contact of the user with the medical device by detecting and classifying a non-contact gesture command of the user. At least one processor receives a command signal at the medical device, in which the command signal applies to the treatment being performed using the medical device, and in which the command signal corresponds to the non-contact gesture command of the user. At least one processor processes the command signal at the medical device to generate information corresponding to the treatment performed using the medical device. At least one processor applies the information to adjust the medical device. The medical device may include a dialysis machine, and the information applied to adjust the medical device may include information that modifies dialysis treatment information displayed during a dialysis treatment. The E-field device may be integrated with the medical device, and the command signal may be integrally communicated by the E-field device to the medical device in response to the non-contact gesture command of the user. The E-field device may be implemented on a remote interface device that transmits a wireless signal to the medical device in response to the non-contact gesture command of the user. The non-contact gesture command may cause a change in the information applied to adjust the medical device, and the change in the information may include a different screen being displayed on the medical device, a section of the information being activated by the non-contact gesture command and causing a change in operation of the dialysis machine and/or a displayed cursor being illuminated by proximity detection of a hand and a location of the cursor manipulated by a position of the hand. The E-field device may include an E-field sensor that detects changes in electric field by a hand or finger of a user intruding a sensing area of the E-field sensor that detects the non-contact gesture command of the user and a gesture command recognition device that classifies the non-contact gesture command and identifies an action to be performed at the medical device.

### BRIEF DESCRIPTION OF THE DRAWINGS

Embodiments of the system described herein are explained with reference to the several figures of the drawings, which are briefly described as follows.

FIG. 1 is a schematic illustration of an example of a patient care environment in which a patient seated in a chair receives medical treatment from a dialysis machine and which may be used in connection with an embodiment of the system described herein.

FIG. 2 is a schematic illustration of another example of a patient care environment that may be used in connection with an embodiment of the system described herein.

FIG. 3 is schematic illustration of an example implementation of the dialysis machine according to an embodiment of the system described herein.

FIG. 4 is a schematic illustration of a more detailed implementation of the dialysis machine according to an embodiment of the system described herein.

FIG. 5 is a schematic illustration showing example information that may be displayed on the display of the dialysis machine and that may be navigated using the interface device according to an embodiment of the system described herein.

FIGS. 6A and 6B provide schematic illustrations of an example E-field sensor operating in connection with a dialysis device component, such as a display of a dialysis machine and/or a display of a remote interface device wirelessly coupled to a dialysis machine according to an embodiment of the system described herein.

FIG. 7 is a schematic illustration showing another embodiment of the system described herein in which the display of the dialysis machine may be controlled using E-field based gesture interfacing control in connection with a dialysis treatment being performed in the patient care environment

FIG. 8 is a schematic illustration showing a gesture by a user (e.g., HCP) that may be used to navigate and control one or more screens displayed on the display of the dialysis machine according to an embodiment of the system described herein.

FIG. 9 is a schematic illustration according to an embodiment of the system described herein showing the HCP using an interface device having e-field sensing capability for gesture detection and recognition in connection with the monitoring and/or control of a dialysis machine in connection with a dialysis treatment being performed in the patient care environment.

FIG. 10 is a flow diagram showing processing steps in connection with non-contact control of a dialysis machine by a user with a command input device like that described elsewhere herein, and that specifically may include an E-field sensor for gesture detection and a gesture command recognition device according to an embodiment of the system described herein.

FIG. 11 is a flow diagram showing processing in connection with specific actions of navigating and/or activating screens of a display of a dialysis machine during a dialysis treatment according to an embodiment of the system described herein.

### DETAILED DESCRIPTION OF VARIOUS EMBODIMENTS

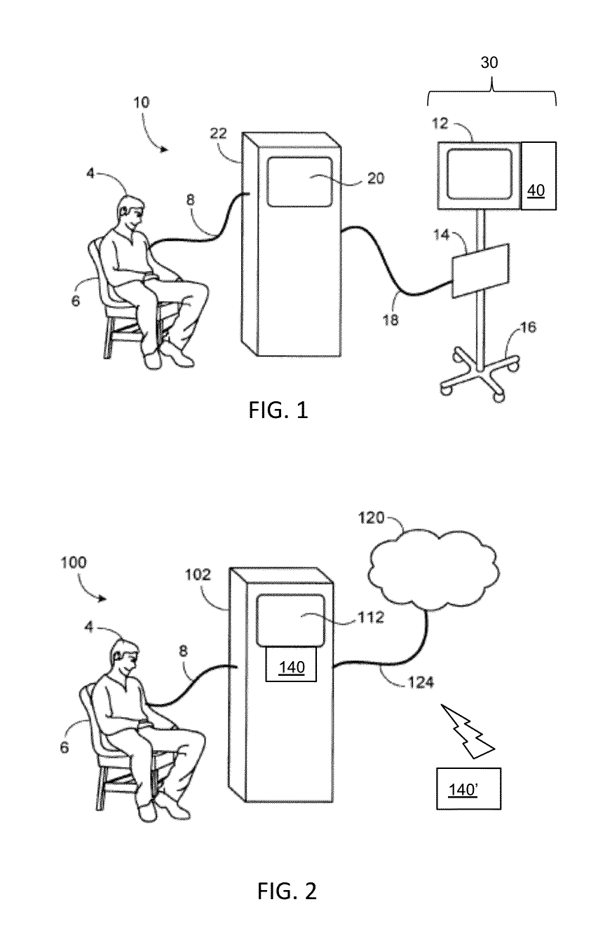

FIG. 1 is a schematic illustration of an example of a patient care environment 10 in which a patient 4 seated in a chair 6 receives medical treatment from a treatment station 22 and which may be used in connection with an embodiment of the system described herein. The medical treatment is, for example, dialysis. The treatment station 22 may be a dialysis treatment station or dialysis machine. A tube or blood line 8 transports blood from the patient 4 to the dialysis machine 22 and back again to the patient 4 after processing and treatment in the dialysis machine 22. The dialysis machine 22 with display 20 may be connected via cabling 18 to a controller device 30 that may include a processor 14 which controls a display 12. In various embodiments, the display 20 may display information corresponding to a dialysis treatment being performed by the dialysis machine 22. The display 12 may be mounted on a movable stand 16 of the controller device 30. The display 12 permits a health care practitioner (HCP), such as a nurse, a patient care technician (PCT), or even a patient, to interface with the display 12 to, for example, to monitor and/or control the dialysis machine 22 and/or to enter patient or other data, for example. The display 12 may include touch screen display capability as well as be implemented in connection with gesture based detection and control capability as discussed in detail herein.

According to various embodiments of the system described herein, a command input device 40 may be coupled to the controller device 30 that may be used to control the dialysis machine 22 in a non-contact manner, as further discussed in detail elsewhere herein. In an embodiment, the command input device 40 may include an electrical field (E-field) sensor with gesture detection and recognition capabilities as further discussed in detail elsewhere herein. Further, the command input device 40 may also include wireless communication capability in connection with wireless coupling to one or more wireless interface devices that may be used by a PCT to monitor and/or control a dialysis treatment being performed by the dialysis machine 22, as further discussed elsewhere herein. Various embodiments for the one or more wireless interface devices and for the actions and functions of the command input device 40 in connection with control of the dialysis machine 22 are further discussed in detail elsewhere herein. It is noted that where reference is made herein to wireless coupling, the system described herein may be used with any appropriate wireless communication technology, including, for example, IEEE 802.11b/g, 802.11b/g/n, and/or Bluetooth, having appropriate security and encryption standards, and used in conjunction with appropriate wireless networks, having hardware and software components, that support such wireless communication technologies.

FIG. 2 is a schematic illustration of another example of a patient care environment 100 that may be used in connection with an embodiment of the system described herein. In the patient care environment 100, the patient 4 is seated in the chair 6 and receives medical treatment from a treatment station, such as a dialysis machine 102. The tube or blood line 8 is used for transporting blood from the patient 4 to the dialysis machine 102 and back again to the patient 4 after processing and treatment of the blood in the dialysis machine 102. The dialysis machine 102 may be configured to communicate with an external network 120, such as a local-area network or the Internet, via a wired or wireless connection 124. The network 120 may include one or more databases or other stores of information that securely contain medical information that may be accessed in connection with operation of the system described herein. It is noted that the system described herein may be used in connection with dialysis products produced by Fresenius Medical Care North America of Waltham, Mass., including, for example, Fresenius hemodialysis systems (e.g., a 2008T system).

In an embodiment, the dialysis machine 102 may include a display 112. The dialysis machine 102 may centralize and consolidate dialysis functions and data entry functions in a single device 102, without, e.g., the use of a separate, external display (e.g., display 12 of FIG. 1) or a separate, external processor (e.g., processor 14) with associated equipment (e.g., movable stand 16). In an embodiment, the dialysis machine 102 may include one or more processors 114, like the processor 14, that may be used in connection with interfacing with, and control of, the dialysis machine 102, for example, by an HCP during a dialysis treatment. Consolidation of functions in a single dialysis machine 102 may advantageously reduce the amount of external cabling (e.g., cabling 18) to the device 102. The dialysis machine 102 may further reduce the amount of space needed for dialysis treatment and present less crowding of the patient care environment 100. An HCP may be able to focus solely on the dialysis machine 102, or the display 112 of the dialysis machine 102, without the HCP's attention being diverted to, e.g., another external display. The dialysis machine 102 may reduce power consumption and cost as compared to other, non-centralized implementations.

In an embodiment, a command input device 140 may be coupled to the dialysis machine 102. In an embodiment, the command input device 140 may be an E-field sensor with gesture command recognition capability as further discussed in detail elsewhere herein. As noted in connection with the command input device 40 of FIG. 1, and as discussed in detail elsewhere herein, the command input device 140 may also include wireless channel components that may be used in connection with receiving external or remote signals that may be used to control the dialysis machine 102 and/or may be transmit signals in connection with operation of the dialysis machine 102. In another embodiment, a command input device 140′, that may be like the command input device 140, but may be separate or remote from the dialysis machine 102 and coupled wirelessly thereto. Further, in an embodiment, the command input device 140′ may also be wirelessly coupled to the network 120. Accordingly, in various embodiments, functions of the command input device 140′ may include control of and/or information exchange with the dialysis machine 102 via direct communication therewith and/or the command input device 140′ may interface with the dialysis machine 102 via the network 120. Further features and functions of the command input devices 140 and 140′ are discussed in detail elsewhere herein.

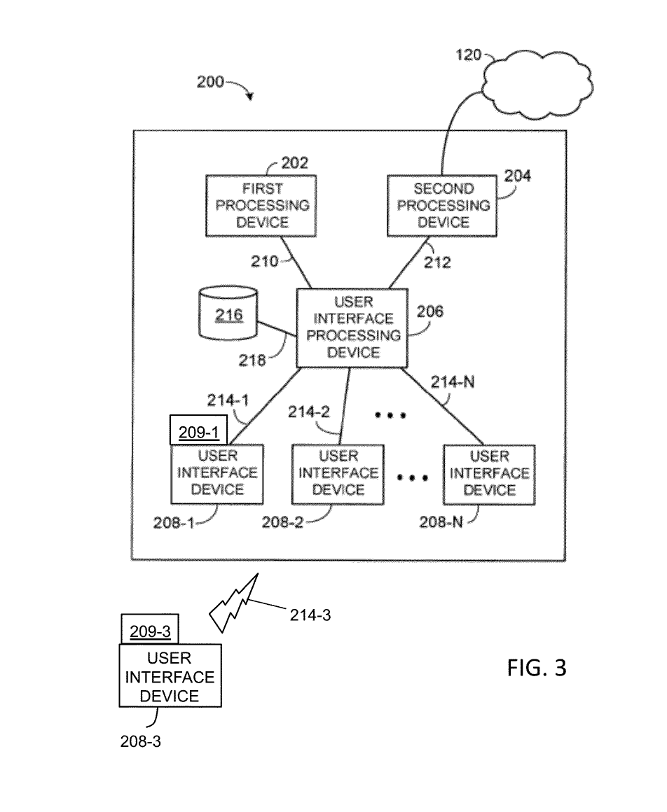

FIG. 3 is schematic illustration of an example implementation 200 of the dialysis machine 102 according to an embodiment of the system described herein. A user interface processing device (UIP) 206 may be configured to share user interface resources, i.e., user interface devices 208-1, 208-2, 208-3 . . . , 208-N, between a first processing device 202 and a second processing device 204. Both the first and the second processing devices 202, 204 may be connected to the UIP 206 via respective connections 210, 212, while the user interface devices 208-1, 208-2, 208-3 . . . , 208-N are connected to the UIP 206 via connections 214-1, 214-2, 214-3 . . . , 214-N. Although one UIP 206 is shown in FIG. 3, several user interface processing devices may be used to implement the functionality of the UIP 206. The UIP 206 is connected to memory 216 via a connection 218. Other memory (not shown) may be connected to, and, used by, e.g., the first processing device 202 and/or the second processing device 204. The second processing device 204 of the device 200 may be configured to communicate with the external network 120, such as a local-area network or the Internet, via a wired or wireless connection 124 (and, e.g., via a network interface (not shown)). In other implementations, other processing devices such as the UIP 206 or the first processing device 202 may communicate with an external network such as the external network 120.

The user interface devices 208-1, 208-2, 208-3 . . . , 208-N may include any of a variety of user interface devices known in the art, such as an alphanumeric keyboard or a keypad, a pointing device (e.g., a touchpad, a mouse, or a trackball), a display with a touch screen, and a display that enables electrical field (E-field) sensing for gesture detection as a control input as according to the system described herein and as further discussed in detail elsewhere herein. In an implementation, one or more of the user interface devices 208-1, 208-2, 208-3 . . . , 208-N may be located external to the device 200 and coupled via wired and/or wireless connections to the device 200. Specifically, for example, user interface device 208-3 is shown remotely located and wirelessly coupled, via wireless connection 214-3, to the device 200. Various embodiments for a user interface device, like that of user interface device 208-3, that may be used to wirelessly monitor and/or control the dialysis machine 102 are further discussed in detail elsewhere herein and may include use of a remotely located E-field sensing device that detects and processes gesture input of a user through E-field sensing and is wirelessly coupled to the dialysis machine 102 to provide wireless signals to control the display 112.

As described herein, the UIP 206 may be configured to share the user interface devices 208-1, 208-2, 208-3 . . . , 208-N between the first processing device 202 and the second processing device 204. The UIP 206 may switch focus from the first processing device 202 to the second processing device 204. The UIP 206 may likewise switch focus from the second processing device 204 to the first processing device 202. Specifically, a processing device, such as the first or the second processing device 202, 204 of FIG. 3, may be said to have focus when the processing device has control of, and/or is controlled by, one or more user interface devices connected to, or communicating with, the processing device (e.g., via one or more user interface processing devices). That is, in this example, when a processing device has focus, a user interface device connected to, or communicating with, the processing device (e.g., via one or more user interface processing devices) will generally affect operation of the processing device, and thereby the dialysis machine 102. User interactions with a user interface device will likewise generally affect operation of the processing device in this instance. Likewise, in this example, when a processing device has focus, the processing device may control a user interface device (such as a video display) connected to, or communicating with, the processing device (e.g., via one or more user interface processing devices).

When a processing device, such as the first or the second processing device 202, 204 of FIG. 3, does not have focus, then, for example, the processing device may not have control of and/or be controlled by one or more user interface devices connected to, or communicating with, the processing device (e.g., via one or more user interface processing devices). Rather, another processing device may have been given focus. One or more user interface processing devices such as the UIP 206 may send protocol data to the processing device, even when the processing device does not presently have focus, so that the processing device may be configured to maintain connections with one or more user interface devices. That is, from the perspective of the processing device, even when the processing device does not have focus, the processing device may have a connection maintained with a user interface device that the processing device does not control and/or that is not controlled by the processing device when the processing device does not have focus. The UIP 206 may therefore send protocol data related to the one or more user interface devices to the first and the second processing devices 202, 204, irrespective of which processing device 202, 204 has focus.

When a processing device (such as the first processing device 202 or the second processing device 204) has focus, one or more user interface processing devices (such as the UIP 206) may manage communications between one or more user interface devices (such as the user interface devices 208-1, 208-2, 208-3 . . . , 208-N) and the processing device. The UIP 206 may, when the processing device has focus, permit the user interface devices 208-1, 208-2, 208-3 . . . , 208-N to affect operation of the processing device. The UIP 206 may switch between modes. The modes may be exclusive of one another and may include a mode in which the first processing device 202 has focus, and a mode in which a second processing device 204 has focus.

According to various embodiments of the system described herein, one or more of the interface devices 208-1 to 208-N may include gesture control features that enable gestures of a user to be used to control the dialysis machine 102. Interface device 208-1 is shown with a gesture detection device 209-1 that may provide for a user to control the dialysis machine 102, and/or display 112 thereof, using contactless gestures. In particular, the gesture detection device 209 may be an E-field sensor gesture detection device that may recognize and process gesture input and commands of a user based on E-field sensing, as further discussed elsewhere herein. Similarly, the interface device 208-3 is shown with a gesture detection device 209-3 that may be used for gesture detection and processing in connection with control input, as discussed in detail elsewhere herein. Additionally, one or more remote interface devices, such as interface device 208-3, as illustrated, may be wirelessly coupled to the dialysis machine 102 via wireless components thereof, such as wireless transmitting/receiving components of the command input devices 40, 140 or 140′ discussed in FIG. 1 and/or FIG. 2.

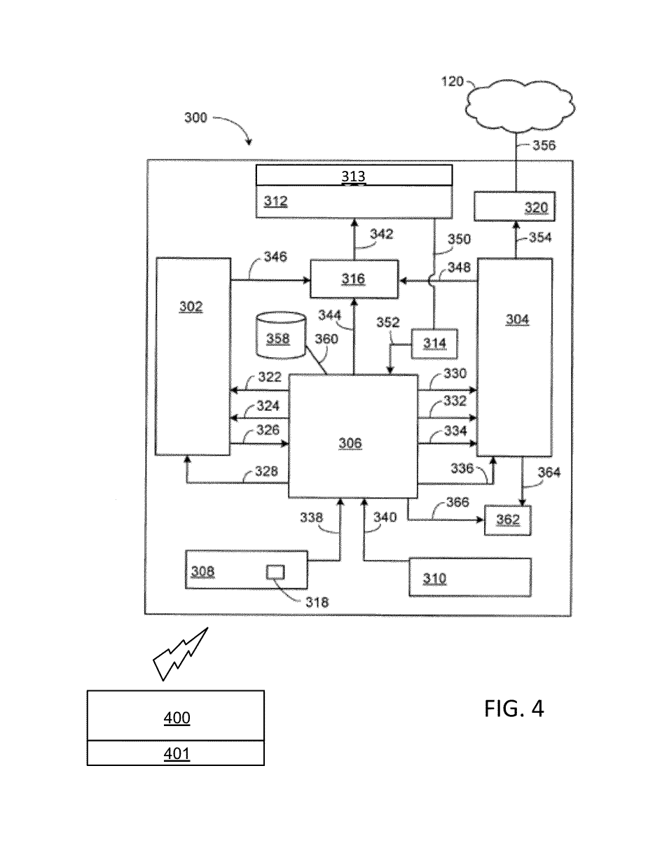

FIG. 4 is a schematic illustration of a more detailed implementation 300 of the dialysis machine 102 according to an embodiment of the system described herein. A UIP 306 is configured to share user interface resources, such as a keyboard 308 and a pointing device 310 (such as a touchpad) between a first processing device 302 and a second processing device 304. Further user interface components, according to the system described herein, may include a display 312, like the display 112 discussed herein, that may be coupled to or otherwise integrated with an E-field gesture detection controller 313, as discussed in detail elsewhere herein. In various embodiments, the display 312 may also further include touch screen input control features, such as touch screen controller 314. In other embodiments, the system described herein may operate in connection with use of a remote interface device 400 that may include an E-field gesture detection controller 401 to receive user gesture input and wirelessly transmit corresponding control signals to the dialysis machine 102 for control of the display 112/312 thereof, as further discussed in detail herein. It is noted that the first processing device 302 may be a functional dialysis processing device (FHP) 302 that may be configured to monitor dialysis functions of the device 300. The second processing device 304 may be a microprocessor, such as a standard personal computer (PC) processor, embedded within the device 300, and may be referred to as an embedded processing device (EP) 304. The FHP 302 is connected to the UIP 306 via connections 322, 324, 326, 328, and the EP 304 is connected to the UIP 306 via connections 330, 332, 334, 336.

The keyboard 308 is connected to the UIP 306 via connection 338. The pointing device 310 is connected to the UIP 306 via connection 340. The display 312 may be connected to a digital video switch 316 via connection 342, which is in turn connected to the UIP 306, the FHP 302, and the EP 304 via respective connections 344, 346, 348. A touch screen controller 314 may be connected to the display 312 via connection 350, and to the UIP 306 via connection 352. Although one UIP 306 is shown in FIG. 4, several user interface processing devices may be used to implement the functionality of the UIP 306. The UIP 306 is connected to memory 358 via a connection 360. Other memory (not shown) may be connected to, and, used by, e.g., the FHP 302 and/or the EP 304. The EP 304, for example, may utilize a flash memory rather than a conventional hard drive. The device 300 also includes an audio device 362. The audio device 362 is connected to the EP 304 via connection 364 and the UIP 306 via connection 366. FIG. 4 is intended to show functional connections between devices of the device 300, so more or fewer connections may be used than are shown in FIG. 4.

As described above, the UIP 306 may switch focus from the FHP 302 to the EP 304. The UIP 306 may likewise switch focus from the EP 304 to the FHP 302. When the FHP 302 has focus, one or more of the keyboard 308, the pointing device 310, the display 312 with a touch screen will generally affect operation of the FHP 302. When the EP 304 has focus, the keyboard 308, the pointing device 310, the display 312, and/or the remote interface device 400 may generally affect operation of the EP 304. User interactions with the devices 308, 310, 312, 400 will likewise generally affect operation of whichever processing device (the FHP 302 or the EP 304) has focus. The processing device that has focus (the FHP 302 or the EP 304) may control, e.g., the display 312 in certain circumstances.

In various implementation, one or more of the user interface devices may be located external to the device 300. In this example implementation, when the EP 304 has focus, the FHP 302 does not have focus, and the FHP 302 may not have control of and/or be controlled by the devices 308, 310, 312, 400. When the FHP 302 has focus, the EP 304 does not have focus, and the EP 304 may not have control of and/or be controlled by the devices 308, 310, 312, 400. The UIP 306 may send protocol data relating to the devices 308, 310, 312 to the EP 304 and the FHP 302, even when one of these devices does not have focus, so that the EP 304 and the FHP 302 may maintain connections with the devices 308, 310, 312. That is, from the perspective of the processing device (EP 304 or FHP 302) that does not have focus, a connection at least appears to be maintained with the devices 308, 310, 312, 400, even though these devices 308, 310, 312, 400 are not controlled by, and do not control, the processing device that does not have focus. The UIP 306 may therefore send protocol data related to the devices 308, 310, 312, 400 to the FHP 302 and the EP 304, irrespective of which processing device 302, 304 has focus. The UIP 306 may switch between modes. The modes may be exclusive of one another and may include a mode in which the first processing device 302 has focus, and a mode in which the second processing device 304 has focus.

The interface device 400 may include one or more indictors, such as lights, that may communicate a successful pairing of the interface device with the command input device 140, of the dialysis machine 102 and/or with the remote command input device 140′ communicating with the dialysis machine 102. The indicators may further communicate other information, such as low battery and/or other information concerning the communication pathway between the interface device 400 and the dialysis machine 102.

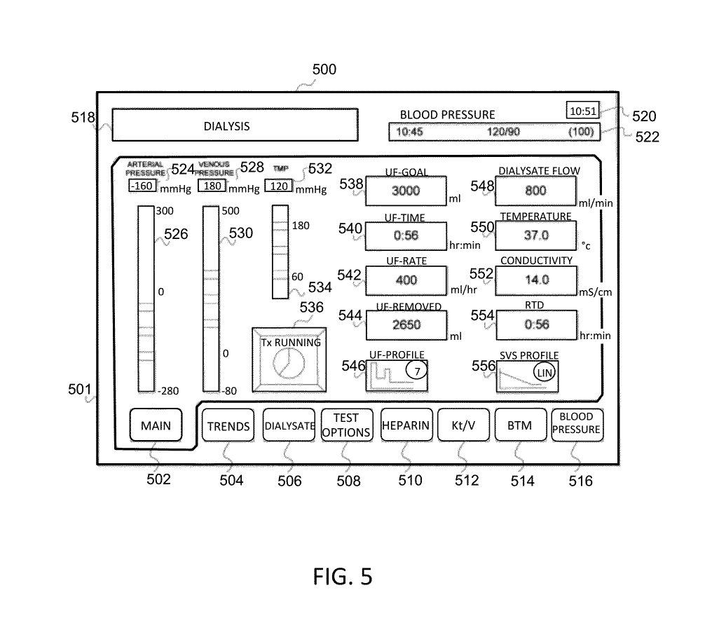

FIG. 5 is a schematic illustration showing an embodiment of information 500 that may be displayed on the display 112 of the dialysis machine 102, that may be navigated using non-contact gesture detection input according to an embodiment of the system described herein. The illustrated embodiment of the information 500 is presented by way of example only, and other information, particularly other operational functions and features for controlling and/or monitoring a dialysis treatment, may be displayed and/or controlled in accordance with the system described herein. In various embodiments, the system described herein may be used in connection with generation and/or display of information corresponding to a medical device, medical treatment and/or patient related data. In the illustrated embodiment, the information 500 may include a treatment screen on the display 112 of the dialysis machine 102 that incorporates the methods and systems for monitoring and/or controlling functions of the dialysis machine 102 that are discussed herein. Other systems and interfaces may also be used for controlling a dialysis machine and/or other medical device, and reference is made, for example, to U.S. Pat. No. 6,775,577 to Crnkovich et al., entitled “Method and System for Controlling a Medical Device,” which is incorporated herein by reference.

Screen access buttons 502 (main access), 504 (trends), 506 (dialysate), 508 (test options), 510 (heparin), 512 (Kt/V), 514 (BTM), and 516 (blood pressure) may be used to access the various treatment screens in a manner that may be similar to that accessed at the display 112. For example, as illustrated, the main access button 502 has been activated, for example based on gesture detection input as discussed in detail herein, revealing a main treatment access screen 501 that may be displayed on the display 112 of the dialysis machine. It is noted that, in other embodiments, different and/or summarized versions of the information displayed on the display 112 of the dialysis machine 102 may be displayed on the interface device 400. A different treatment access screen may be displayed, for example, by pressing the different screen access buttons. The main treatment access screen 501 provides a general overview of the status of the current treatment. Other treatment screens may offer a more in-depth view of specific aspects of the current treatment, though some treatment screens may have some of the same information displayed as found on other treatment screens.

A status box 518 appears at the top left corner of the treatment screen being displayed in the information 500. During normal operation it displays the operation mode of the machine, which in this case is “Dialysis.” During alarm situations, a warning message may be displayed in the status box 518. The message displayed in the status box 518 may also prompt the operator for a specific action in situations when the treatment parameters are being set. During normal treatment, a box 520 displays the current time and the box 522 displays the time of the last blood pressure reading and the patient's blood pressure and pulse rate at that time. Arterial pressure in mmHg is displayed numerically in a meter box 524, and graphically in a bar graph 526. Similarly, venous pressure in mmHg is displayed numerically in a meter box 528 and graphically in a bar graph 530, and transmembrane pressure (TMP) in mmHg is displayed numerically in a meter box 532 and graphically in a bar graph 534.

A Tx clock button 536 may be activated start, or to pause or suspend, the treatment. The Tx clock button 536 controls multiple functions of the hemodialysis machine when it is activated. A UF-goal button 538 displays the desired ultrafiltration (UF) in milliliters to be removed during the dialysis treatment. This is typically the difference between the patient's pre and dry weight plus saline or fluid intake during treatment. The UF-time button 540 acts as a countdown timer displaying the remaining time in hours and minutes that ultrafiltration will be performed. The timer stops during a blood alarm or whenever the UF pump is stopped. During treatment, A UF-rate button 542 displays the current rate of ultrafiltration in milliliters per hour. The rate ultrafiltration occurs is determined by the values entered in a UF-goal button 538 and a UF-time button 540 and the profile selected with a UF-profile button 546. A UF-removed button 544 keeps a running total in milliliters of the fluid drawn from the patient through ultrafiltration. When the value displayed in the UF-Removed button 544 is equal to the value entered in the UF-goal button 538, an alarm sounds and the message, “UF GOAL REACHED” is displayed in the status box 518. A UF-profile button 546 when touched brings up the UF Profile selection screen. Once a profile is selected, and the operator pushes the main access button 502, the profile selected is displayed in the UF-profile button 546.

A dialysate flow button 548 displays the current dialysate flow rate in milliliters per minute. A temperature button 550 displays the current temperature in degrees centigrade of the dialysate. Pressing the temperature button 550 allows the operator to set the desired temperature, and thereafter the actual temperature is displayed. If the temperature varies too far from the set point, an alarm sounds, a warning message is displayed in the status box 518, and the dialysate goes into bypass. A conductivity button 552 displays the current conductivity in millisiemens per centimeter of the dialysate. An RTD (Remaining Time of Dialysis) button 554 acts as a countdown timer displaying the amount of treatment time remaining At the end of treatment (RTD=0:00) an alarm sounds and the message “RTD ZERO” is displayed in the status box 518. An SVS profile button 556 when touched brings up the Sodium Variation System (SVS) profile selection screen. Once a profile is selected, and the operator pushes the main access button 502, the profile selected is displayed in the SVS profile button 556.

In accordance with the system described herein, systems and techniques are detailed for providing control of a medical station, such as a dialysis machine, based on E-field sensing of non-contact gesture input from a user.

An electric field (E-field) is generated by electrical charges and is spread three-dimensionally around a surface carrying the electrical charge. Applying direct voltages (DC) to an electrode results in a constant electric field. Applying alternating voltages (AC) makes the charges and, thus, the field, vary over time. When the charge varies sinusoidally with frequency f, the resulting electro-magnetic wave is characterized by wavelength X=c/f, where c is the wave propagation velocity in vacuum the speed of light. (See, for example, Microchip Technology, Inc., “MGC3130: Single-Zone 3D Tracking and Gesture Controller Data Sheet,” DS40001667C, November 2013, 46 pp., which is incorporated herein by reference, for an explicit description of the E-field sensing principles and example sensors, like that discussed elsewhere herein, that may be used in connection with the system described herein).

Capacitive proximity or E-field sensors are known that may be used to detect gestures from a user's hand or fingers in proximity to a sensing area of the sensor based on operations of transmitter and receiver electrodes of the sensor. Once a user intrudes the sensing area with a hand, the electrical field distribution around the sensing area becomes distorted. The field lines intercepted by the hand are shunted to ground through the conductivity of the human body itself. The proximity of the hand causes a compression of the equipotential lines and shifts receiver electrode signal levels of to a lower potential which may be detected by E-field sensor. For illustrative discussion of examples of sensors that may be used to detect 3D gestures of a user, reference is made, for example, to U.S. Pub. No. 2013/0155010 A1 to Curtis et al., entitled “Capacitive Proximity Based Gesture Input System,” U.S. Pub. No. 2013/0249855 A1 to Zhang, entitled “System and Method to Share Electrodes Between Capacitive Touch Controller and Gesture Detection Device,” U.S. Pat. No. 7,358,742 B2 to Cehelnik, entitled “DC & AC Coupled E-field Sensor,” and U.S. Pat. No. 8,514,221 B2 to King et al., entitled “Working with 3D Objects,” which are all incorporated herein by reference.

By way of example only, an E-field sensing product that may be used in connection with an embodiment the system described herein is Microchip's GestIC® technology which utilizes E-fields for advanced proximity sensing and allows realization of user interface applications by detection, tracking and classification of a user's hand or finger gesture motions in free-space. The GestIC® technology uses transmit frequencies in the range of 100 kHz, which reflects a wavelength of about three kilometers. With electrode geometries that may be less than twenty by twenty centimeters, this transmit wavelength is much larger in comparison. Therefore, the magnetic component is practically zero and no wave propagation takes place. The result is a quasi-static electrical near field that can be used for sensing conductive objects such as the human body. Microchip's GestIC® technology utilizes thin sensing electrodes made of a conductive material and may be integration behind a display device's housing. The technology may be used with Microchip's Colibri Gesture Suite that provides a library of GestIC® technology features. The Colibri Suite uses a Hidden Markov Model based gesture recognition engine in conjunction with x/y/z hand-position vector post-processing and provides a level of user-independent recognition of 3D hand and/or finger gestures. The Microchip's GestIC® technology has a 0 to 15 cm detection range.

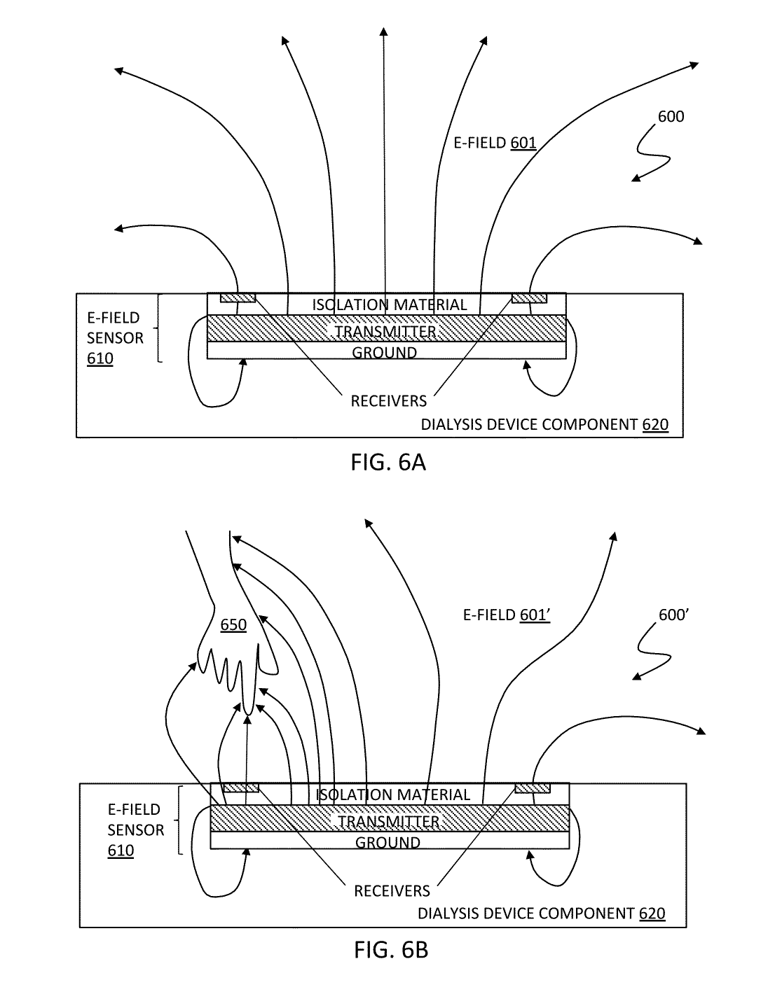

FIGS. 6A and 6B provide schematic illustrations 600, 600′ of an example E-field sensor 610 operating in connection with a dialysis device component 620, such as a display of a dialysis machine and/or a display of a remote interface device wirelessly coupled to a dialysis machine according to an embodiment of the system described herein. In an embodiment, the E-field sensor 610 may be a Microchip's GestIC® product. FIG. 6A shows the illustration 600 in which an E-field 601 of the E-field sensor 610 is undistorted. The E-field sensor 610 includes transmitter and receiver components separated by an isolation material and shown atop a grounded layer. FIG. 6B shows the illustration 600′ showing a distorted E-field 601′ after a hand/finger of a user 650 intrudes into the electrical field. As shown, the field lines are drawn to the hand due to the conductivity of the human body and shunted to ground. The E-field sensor 610 may detect the E-field variations at different positions to measure the origin of the E-field distortion and this information is used to calculate the position of the user's hand, track movements and identify gestures.

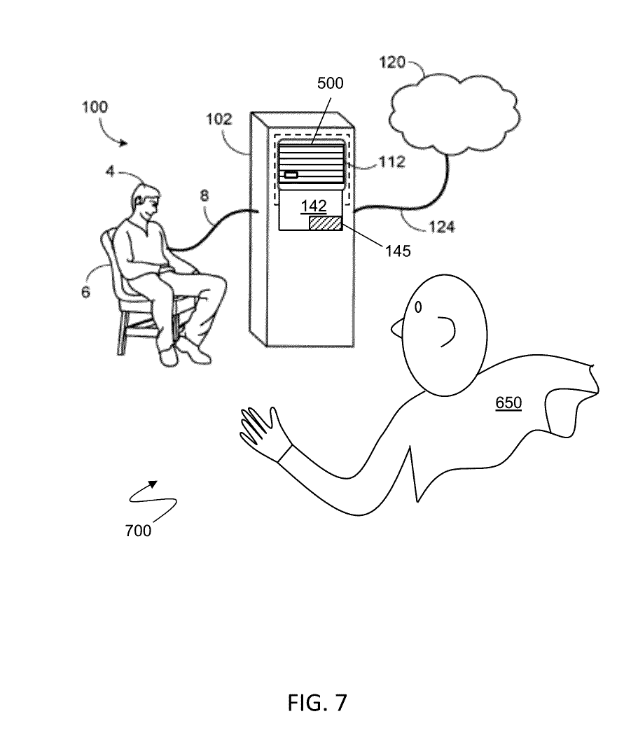

FIG. 7 is a schematic illustration 700 showing another embodiment of the system described herein in which the display 112 of the dialysis machine 102 may be controlled using E-field based gesture interfacing control in connection with a dialysis treatment being performed in the patient care environment 100 (see, e.g., FIG. 2). In this embodiment, an E-field sensor 142 that is coupled to the dialysis machine 102 may include and/or be coupled with a gesture command recognition device 145 that may recognize and process gestures by the HCP 650 as input for controlling the device. The E-field sensor 142 may be a sensor that detects 3D gestures by a user based e-field changes, as further discussed elsewhere herein, and the command recognition device 145 may include hardware and/or software components that operate to recognize the gesture as a specific command of the user (e.g., in connection with navigating and/or activating screens of the display 112). The E-field sensor 142 may be an implementation of the command input device 140, discussed previously, and in connection with the user interface device 208-1 and the gesture detection device 209, discussed previously, and may operate to recognize and interprets non-contact commands of the HCP 650 in connection with non-contact gesture-based selection, control and activation of elements of the information 500 being displayed on the display 112 of the dialysis machine 102. In this way, the HCP 650 does not need to re-glove each time an action needs to be taken with respect to the dialysis machine 102, such as modifying a parameter thereof during the dialysis treatment. In an embodiment, the E-field sensor 142 and gesture command recognition device 145 may be implemented using a Microchip's GestIC® product with Colibri Gesture Suite. Accordingly, in an embodiment, the proximity of the user's hand for the gesture control may be within 15 cm of the E-field sensor 142/display 112.

The E-field sensor 142 and gesture command recognition device 145 may be implemented according to various embodiments which may include being incorporated entirely, or in part, into the display 112 of the dialysis machine 102. In other embodiments, the E-field sensor 142/command recognition device 145 may be otherwise disposed elsewhere on the dialysis machine 102 in a manner that suitably facilitates the gesture sensing and command recognition operations and may be appropriately coupled to the display 112. Additionally and/or alternatively, in another embodiment, the E-field sensor 142 and gesture command recognition device 145 may be incorporated in connection with a display of a control unit that is coupled to, but located distally or remotely from, the dialysis machine 102, such as being implemented as the command input device 40 of the display 12 of the controller device 30 (see, e.g., FIG. 1).

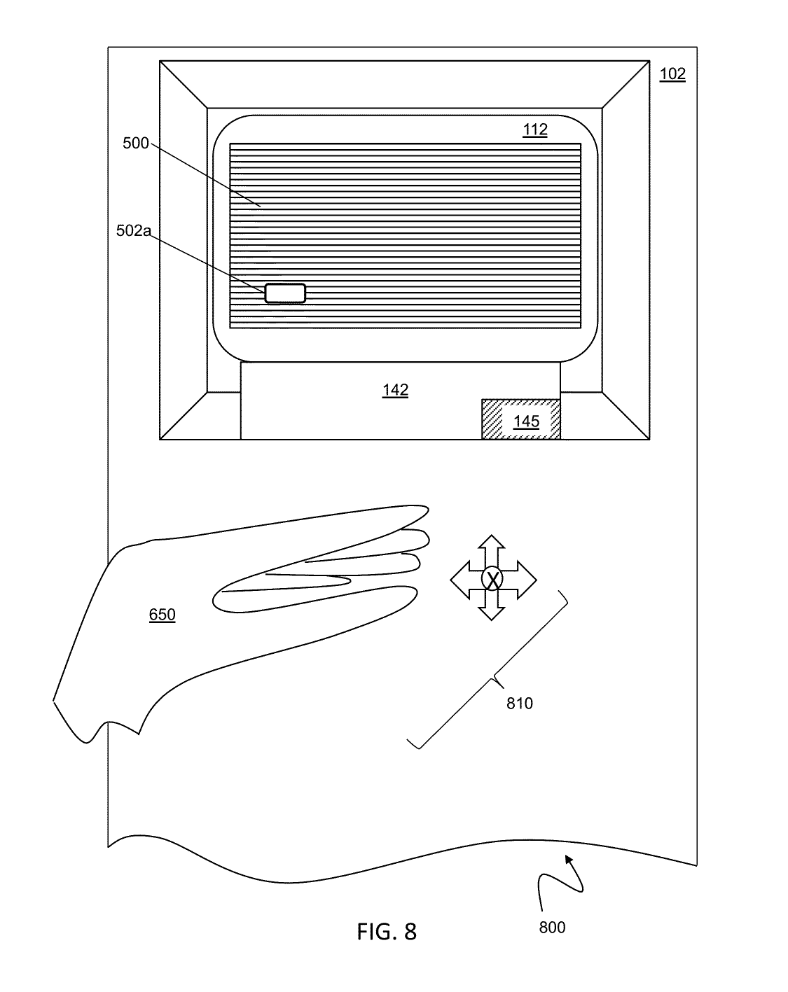

FIG. 8 is a schematic illustration 800 showing a gesture 810 by a user (e.g., HCP 650) that may be used to navigate and control one or more screens displayed on the display 112 of the dialysis machine 102 according to an embodiment of the system described herein. The gesture 810 is recognized by the command recognition device 145 of the E-field sensor 142 coupled to display 112 of the dialysis machine 102. The display 112 shows the information 500 in which a section 502a has been activated by the gesture 810, as sensed by the E-field sensor 142 and recognized and processed by the command recognition device 145. As illustrated, the gesture may include gesture components in three-dimensions (3D) that are sensed by the E-field sensor 142 and recognized and processed by the command recognition device 145. Tracking of the gesture 810 detected by the E-field sensor 142 and recognized by the command recognition device 145 may be visually presented on the display 112, for example, as mouse cursor that tracks the position of the hand. The gesture 810 (and/or further gestures, such as a for example quick forward motion) may activate the section 502a that the cursor is hovering over. For example, visual feedback to the user could be implemented by a changing color of the highlighted section as the user's finger moves forward towards the gesture detection device (E-field sensor 142/command recognition device 145). The activated section 502a may be a button, such as the main access button 502 that activates the providing of a main access treatment screen 501 to provide a general overview of the status of a current dialysis treatment being performed. The providing of the main access treatment screen 501 may be performed by the activation instruction of the button 502 according to the interpretation of the gesture 810 by the user that is processed by the E-field sensor 142/command recognition device 145.

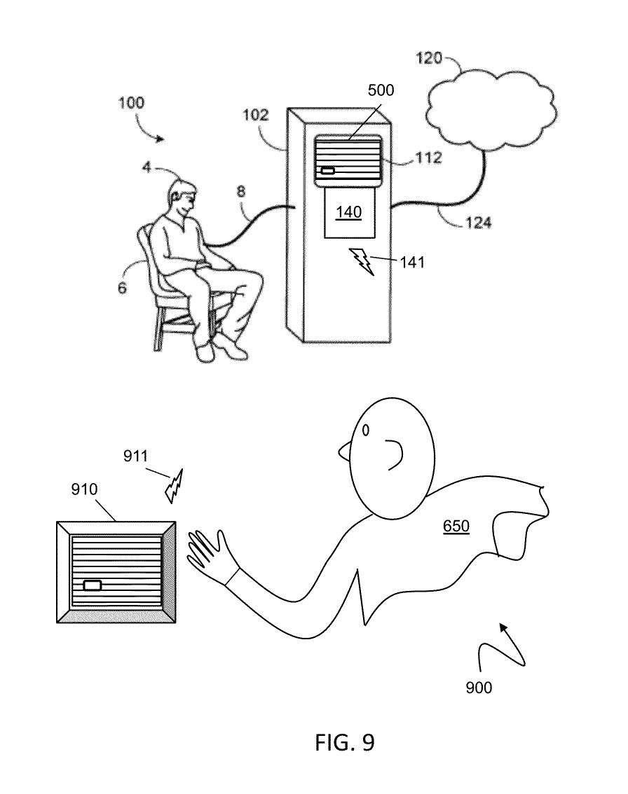

FIG. 9 is a schematic illustration 900 according to an embodiment of the system described herein showing the HCP 650 using an interface device 910 having e-field sensing capability for gesture detection and recognition in connection with the monitoring and/or control of a dialysis machine in connection with a dialysis treatment being performed in the patient care environment 100 (see, e.g., FIG. 2). In this embodiment, the HCP 650 is shown in proximity to the interface device 910 which may be an implementation of the interface device 400/401 and/or the command input device 140′, discussed previously herein. The interface device 910 enables the HCP 650 to control the dialysis machine 102 during the dialysis treatment in a non-contact manner without requiring the HCP to touch and/or otherwise contact the dialysis machine 102 or component thereof. The interface device 910 may include a display with an E-field sensor and gesture command recognition device like that further discussed in detail elsewhere herein.

By the non-contact gestures in proximity to the interface device 910, the HCP may navigate through screens being displayed on the display of the interface device 910 and/or select or activate portions of the information 500 being displayed. In an embodiment, the interface device 910 may be wirelessly coupled to the dialysis machine 102, and specifically to the display 112 of the dialysis machine 102, in which content for display on the display 112 of the dialysis machine 102 is also, or instead, displayed on the display of the interface device 910. The wireless channel for wireless signal transmission and reception to/from the interface device 910 is shown schematically as wireless channel 911 of the interface device 910. It is noted that the information displayed on the interface device 910 may not necessarily be identical to the information displayed on the display 112, but instead may be summarized or condensed version thereof, for example.

Further shown schematically is wireless channel 141 that may be a wireless channel for transmission of signals from wireless channel components of the command input device. For example, over the wireless channel pairing signals and/or acknowledge signal may be communicated among the command input device 140 and the interface device 910, as well as information corresponding to the display of information, and control of the information, of the dialysis machine 102 in accordance with the embodiments discussed herein. It is noted that the wireless communication channel between the interface device 910 and the command input device 140 may include suitable relay components for relaying the signal via the network 120, that may be an internal network and/or an external network, such as the Internet, in connection with operation of the system for the described embodiment(s). Using the interface device 910, the HCP 650 may control the dialysis machine during the dialysis treatment without contacting the display 112 of the dialysis machine 102 and/or without necessarily contacting the interface device 910. In this way, the HCP does not need to re-glove each time an action needs to be taken with respect to the dialysis machine 102, such as modifying a parameter thereof during the dialysis treatment.

In various embodiments, gesture commands recognized by the E-field sensing and gesture detection components of the interface device 910 may be used to control functionality of the treatment screen being displayed as information 500 on the interface device 910. Accordingly, the mechanism of control of the treatment screen may deviate from control of the treatment screen that is being displayed on the display 112 of the dialysis machine 102. For example, whereas the treatment screen displayed on the screen of the interface device 910 may be controlled, for example, by the command-based recognition that may be used to iterate through and/or highlight different buttons of the information 500 for the treatment screen that is being displayed on the screen 410. As discussed elsewhere herein, in other embodiments, the information 500 being displayed on the interface device 910 may present a treatment screen that is somewhat different from the treatment screen presented on the display 112 of the dialysis machine 102 in a manner that facilitates that command-based recognition control enabled by the interface device 910.

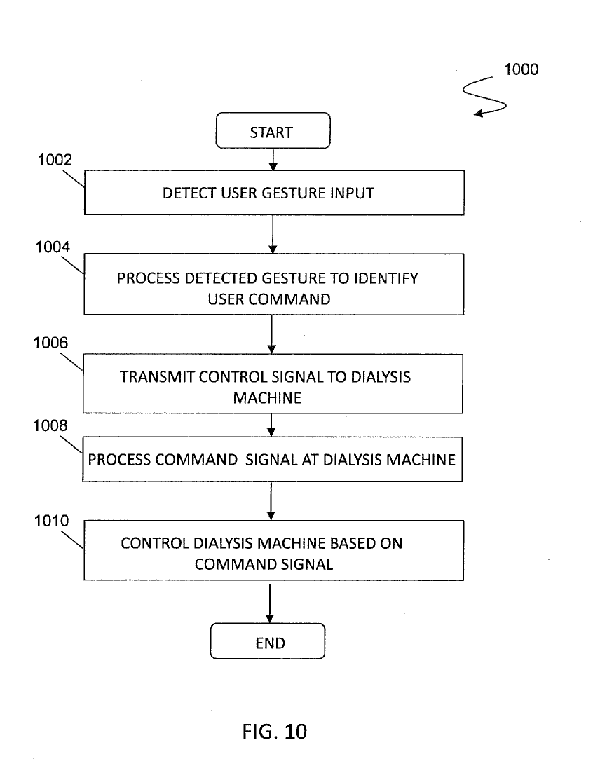

FIG. 10 is a flow diagram 1000 showing processing steps in connection with non-contact control of a dialysis machine by a user with a command input device like that described elsewhere herein, and that specifically may include an E-field sensor for gesture detection and a gesture command recognition device according to an embodiment of the system described herein. At a step 1002, in response to a user (HCP) gesture, the E-field sensor of the command input device detects the gesture of the user according to the capacitive proximity processing and/or other E-field detection techniques, as further discussed elsewhere herein. After the step 1002, at a step 1004, the detected gesture of the user is processed by a gesture recognition device to recognize and interpret the command indicated by the gesture. After the step 1004, at a step 1006, the command input device transmits a control signal to be processed by the display of the dialysis machine that is performing a dialysis treatment on a patient. In an embodiment, the command input device may be integrated with the display of the dialysis machine and controlled by an HCP who is monitoring the dialysis treatment and/or the command input device may be a device remote from the dialysis machine that has the E-field gesture detection and recognition capability and after processing the gesture wirelessly communicates an command signal to the dialysis machine for control thereof. Accordingly, with the system described herein, the HCP does not physically contact the display or other components of the dialysis machine. After the step 1006, at a step 1008, the control signal transmitted by the command input device, that is either integrated with the display or remote from the display of the dialysis device, is processed by the dialysis machine in connection with control of one or more screens displayed on the display of the dialysis machine during the dialysis treatment.

After the step 1008, processing proceeds to a step 1010 where information displayed on the display of the dialysis machine during the dialysis treatment is modified based on the control signal received. For example, as discussed elsewhere herein, the control signal may enable navigation through screens displayed on the display of the dialysis machine and/or activation thereof to control the dialysis machine. After the step 1008, processing is complete for the described processing iteration of the interface device. It is noted that the processing of the flow diagram 1000 may be an on-going process in which the command input device continuously operates to receive and process user gestures. It is noted that the processing steps performed in the flow diagram 1000 may be performed in connection with the execution of software on a non-transitory computer-readable medium by one or more processors of the command input device, the dialysis machine, and/or other appropriate device of the system. In an embodiment, the software may correspond to software that facilitates and/or otherwise interfaces with the dialysis machine in connection with the performance of the dialysis treatment, such as by providing one or more dialysis treatment screens.

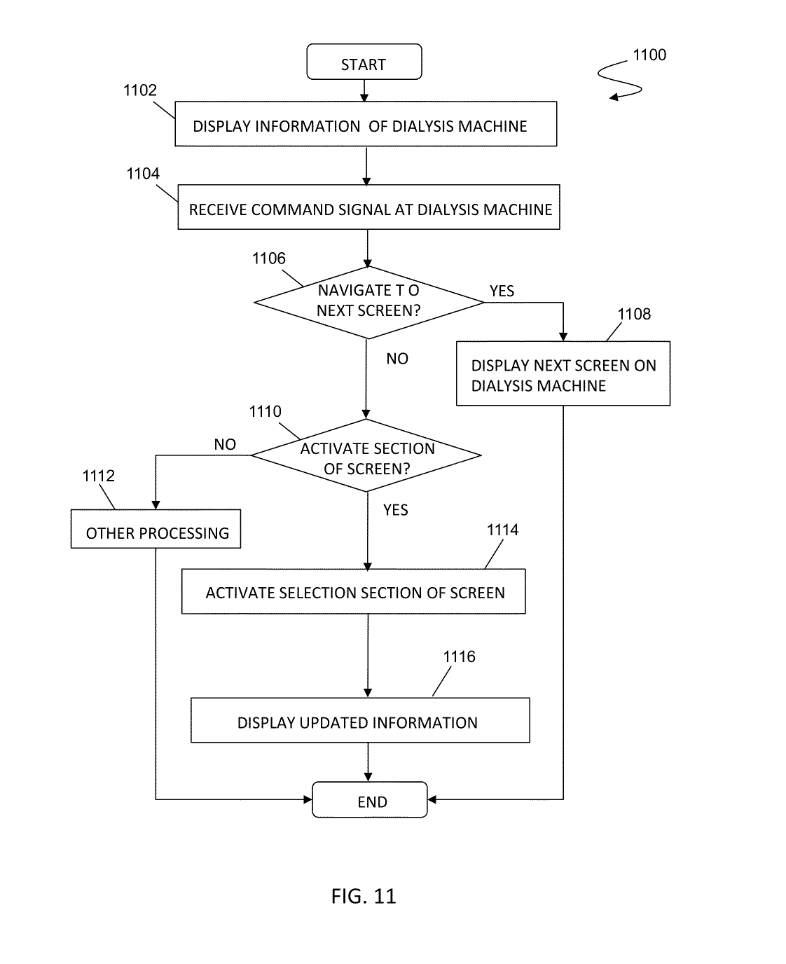

FIG. 11 is a flow diagram 1100 showing processing in connection with specific actions of navigating and/or activating screens of a display of a dialysis machine, like the dialysis machine 22 or 102, during a dialysis treatment according to an embodiment of the system described herein. At a step 1102, a screen is displayed on the display with information concerning operation of the dialysis machine. After the step 1102, processing proceeds to step 1104 where a command signal is received/processed at the dialysis machine corresponding to operation of the dialysis machine. For example, the command signal may be derived from a non-contact gesture input command of a user that is detected and recognized, such as via a E-field sensor and gesture detection and recognition component thereof, and from which the command signal is generated and/or the command signal may be transmitted via a wireless signal sent from a remote command input device, such as the interface device 400, that detects and processes a user gesture at the remote command input device and then wirelessly transmits a control signal to the dialysis machine.

After the step 1104, processing proceeds to a test step 1106 where it is determined whether the command signal received includes instructions to navigate from a current screen of information to a different screen of information displayed on the display of the dialysis machine. If, at the test step 1106, it is determined that input command is to navigate to a different screen, then processing proceeds to a step 1108 where a next screen of information is displayed on the display of the dialysis machine. After the step 1108, processing is complete for the current iteration of the process being discussed, noting that the processing of the flow diagram 1100 may be repeatedly performed.

If, at the test step 1106, it is determined that the input command is not directed to navigating to a different screen, then processing proceeds to a test step 1110 where it is determined whether the input command is directed to activation of a section of the current screen being displayed on the display of the dialysis machine. If, not, then processing proceeds to a step 1112 where other processing is performed with respect to the command signal, and thereafter, processing is complete. If, at the test step 1110, it is determined that the command signal is to select and/or activate a section of the current screen, such as a button displayed on the screen in connection with a dialysis treatment screen, then processing proceeds to a step 1114, where the command signal is processed to select/activate the appropriate section.

After the step 1114, processing proceeds to a step 1116 where the selected portion of the screen is activated (e.g., button on the display is activated) and the current screen information is updated to reflect the activated portion. For example, the received control activation command may have adjusted a parameter of the dialysis treatment being performed and the confirmation is updated information of the dialysis treatment that is transmitted to the interface device. The updated information may therefore correspond to a treatment screen of the dialysis treatment displayed on the dialysis machine. After the step 1114, processing is complete for the current iteration of the processing being described. It is noted that the processing of the flow diagram 1100 may be an on-going process in which the dialysis machine repeatedly monitors for commands and/or signals in connection with the system described herein. It is noted that the processing steps performed in the flow diagram 1100 may be performed in connection with the execution of software on a non-transitory computer-readable medium of the dialysis machine by one or more processors of the dialysis machine, including, in particular, one or more processors of a sensor of the dialysis machine. In an embodiment, the software may correspond to software that facilitates and/or otherwise interfaces with an interface device specifically in connection with non-contact monitoring and control of the dialysis treatment, such as in connection with the providing of dialysis treatment screens. It is noted that the processing of the flow diagram 1100 may be performed in conjunction with other processing of the dialysis machine, including for example, input of commands directly to the dialysis machine via a touch screen display, for example.

It is noted that the system described herein is discussed principally in connection with the use of dialysis machines and treatments. It is noted that, in other embodiments, the system described herein may also be used in connection with other medical devices where non-contact control of such devices is desirable and may be appropriately performed. It is also noted that the system described herein may be used in connection and conjunction with the features and functions of systems for controlling medical devices like that described in co-pending U.S. patent application Ser. No. 13/804,567 to Wang et al., filed Mar. 14, 2013, and entitled “Wireless Controller to Navigate and Activate Screens on a Medical Device,” and U.S. patent application Ser. No. 13/804,761 to Christensen, filed Mar. 14, 2013, and entitled “Wearable Interface for Remote Monitoring and Control of a Medical Device,” which are both assigned to the same assignee as that of the present application and which are both incorporated herein by reference.

Various embodiments discussed herein may be combined with each other in appropriate combinations in connection with the system described herein. Additionally, in some instances, the order of steps in the flow diagrams, flowcharts and/or described flow processing may be modified, where appropriate. Further, various aspects of the system described herein may be implemented using software, hardware, a combination of software and hardware and/or other computer-implemented modules or devices having the described features and performing the described functions. The system may further include a display and/or other computer components for providing a suitable interface with a user and/or with other computers.

Software implementations of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media such as a CD-ROM, a DVD-ROM, an SD card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method for providing non-contact electric field (E-field) gesture interfacing with a medical device, comprising:
operating an E-field device to enable non-contact gesture interfacing with the medical device by a user without physical contact of the user with the medical device by detecting and classifying a non-contact gesture command of the user;
receiving a command signal at the medical device, wherein the command signal applies to a treatment being performed using the medical device, and wherein the command signal corresponds to the non-contact gesture command of the user;
processing the command signal at the medical device to generate information corresponding to the treatment performed using the medical device; and
applying the information to adjust the medical device.

2. The method according to claim 1, wherein the medical device includes a dialysis machine, and wherein the information applied to adjust the medical device includes information that modifies dialysis treatment information displayed during a dialysis treatment.

3. The method according to claim 1, wherein the E-field device is integrated with the medical device, and wherein the command signal is integrally communicated by the E-field device to the medical device in response to the non-contact gesture command of the user.

4. The method according to claim 1, wherein the E-field device is implemented on a remote interface device that transmits a wireless signal to the medical device in response to the non-contact gesture command of the user.

5. The method according to claim 1, wherein the non-contact gesture command causes a change in the information applied to adjust the medical device.

6. The method according to claim 5, wherein the change in the information includes at least one of: (i) a different screen being displayed on the medical device, (ii) a section of the information being activated by the non-contact gesture command and causing a change in operation of the dialysis machine, or (iii) a displayed cursor being illuminated by proximity detection of a hand and a location of the cursor manipulated by a position of the hand.

7. The method according to claim 1, wherein the E-field device includes an E-field sensor that detects changes in electric field by a hand or finger of a user intruding a sensing area of the E-field sensor that detects the non-contact gesture command of the user and a gesture command recognition device that classifies the non-contact gesture command and identifies an action to be performed at the medical device.

8. A non-transitory computer-readable medium storing software for providing non-contact electrical field (E-field) gesture interfacing with a medical device, comprising:
executable code that operates an E-field device to enable non-contact gesture interfacing with the medical device by a user without physical contact of the user with the medical device by detecting and classifying a non-contact gesture command of the user;
executable code that receives a command signal at the medical device, wherein the command signal applies to a treatment being performed using the medical device, and wherein the command signal corresponds to the non-contact gesture command of the user;
executable code that processes the command signal at the medical device to generate information corresponding to the treatment performed using the medical device; and
executable code that applies the information to adjust the medical device.

9. The non-transitory computer readable medium according to claim 8, wherein the medical device includes a dialysis machine, and wherein the information applied to adjust the medical device includes information that modifies dialysis treatment information displayed during a dialysis treatment.

10. The non-transitory computer readable medium according to claim 8, wherein the E-field device is integrated with the medical device, and wherein the command signal is integrally communicated by the E-field device to the medical device in response to the non-contact gesture command of the user.

11. The non-transitory computer readable medium according to claim 8, wherein the E-field device is implemented on a remote interface device that transmits a wireless signal to the medical device in response to the non-contact gesture command of the user.

12. The non-transitory computer readable medium according to claim 8, wherein the non-contact gesture command causes a change in the information applied to adjust the medical device.

13. The non-transitory computer readable medium according to claim 12, wherein the change in the information includes at least one of: (i) a different screen being displayed on the medical device, (ii) a section of the information being activated by the non-contact gesture command and causing a change in operation of the dialysis machine, or (iii) a displayed cursor being illuminated by proximity detection of a hand and a location of the cursor manipulated by a position of the hand.

14. The non-transitory computer readable medium according to claim 8, wherein the E-field device includes an E-field sensor that detects changes in electric field by a hand or finger of a user intruding a sensing area of the E-field sensor that detects the non-contact gesture command of the user and a gesture command recognition device that classifies the non-contact gesture command and identifies an action to be performed at the medical device.

15. A system for enabling non-contact electrical field (E-field) gesture interfacing with a medical device, comprising:
the medical device;
an electrical field (E-field) device that enables non-contact gesture interfacing with the medical device by a user without physical contact of the user with the medical device by detecting and classifying a non-contact gesture command of the user;
at least one processor that receives a command signal at the medical device, wherein the command signal applies to the treatment being performed using the medical device, wherein the command signal corresponds to the non-contact gesture command of the user;
at least one processor that processes the command signal at the medical device to generate information corresponding to the treatment performed using the medical device; and
at least one processor that applies the information to adjust the medical device.

16. The system according to claim 15, wherein the medical device includes a dialysis machine, and wherein the information applied to adjust the medical device includes information that modifies dialysis treatment information displayed during a dialysis treatment.

17. The system according to claim 15, wherein the E-field device is integrated with the medical device, and wherein the command signal is integrally communicated by the E-field device to the medical device in response to the non-contact gesture command of the user.

18. The system according to claim 15, wherein the E-field device is implemented on a remote interface device that transmits a wireless signal to the medical device in response to the non-contact gesture command of the user.

19. The system according to claim 15, wherein the non-contact gesture command causes a change in the information applied to adjust the medical device.

20. The system according to claim 15, wherein the E-field device includes an E-field sensor that detects changes in electric field by a hand or finger of a user intruding a sensing area of the E-field sensor that detects the non-contact gesture command of the user and a gesture command recognition device that classifies the non-contact gesture command and identifies an action to be performed at the medical device.
